(19) United States
(12) Patent Application Publication
(10) Pub. No.: US 2008/0160001 A1
(43) Pub. Date: Jul. 3, 2008

(54) Antihypercholesterolemic Formulation with Less Side-Effects

(75) Inventors: Hongping Ye, Huaibei (CN); Zuolin Zhu, San Diego, CA (US); Meg M. Sun, San Diego, CA (US)

Correspondence Address: HAMRE, SCHUMANN, MUELLER & LARSON, P.C., P.O. BOX 2902, MINNEAPOLIS, MN, 55402-0902, US

(73) Assignee: PFICKER PHARMACEUTICALS LTD., Huaibei (CN)

(21) Appl. No.: 11/887,306

(22) PCT Filed: Mar. 28, 2005
(86) PCT No.: PCT/CN2005/000396

§ 371 Date: Feb. 22, 2008

(51) Int. Cl. A61K 38/43; A61K 31/357; A61K 31/351

(52) U.S. Cl. 424/94.1; 514/452; 514/460

(57) ABSTRACT

The present disclosure provides a combined medicament for reducing blood fat with safety and high efficiency, the active ingredients of which include an agent for lowering blood fat such as statins; an agent for protecting and repairing liver such as silymarin; an agent for protecting kidney, heart, blood vessel or muscle such as coenzyme Q10 and various excipients desired for producing medicaments. The present disclosure can safely and reliably lower the concentration of cholesterol in the blood; protect blood vessels of heart and brain and provide them with nutrition; protect and repair other relevant causes of disease which result in the increasing of blood fat; and lower, even eliminate the side effects of the damage of statins on muscle and liver.

### TECHNICAL FIELD

The present invention relates to a multi-components formulation, particularly to a formulation as antihypercholesterolemic medicaments.

### BACKGROUND ART

Blood fat, being an important substance in human body, possesses a lot of very important functions. In recent years, because of the continuous increases in living people's standards, as well as the changes of dietary structures and lifestyles, the level of blood fat in the people increased continuously from year to year in general. It is estimated that about 30-40% of people suffered from abnormal blood fat metabolism out of the critical standard in different degrees, i.e. hyperlipoidemia. hyperlipoidemia mainly means that the levels of cholesterol (TC), triglyceride (TG) and/or low density lipoprotein (LDL) in the serum are too high, and/or the level of high density lipoprotein (HDL) in the serum is too low.

Many research data indicate that hyperlipoidemia may result in cardiovascular and cerebrovascular diseases and microcirculation obstacle, the direct damage of which is to accelerate systemic atherosclerosis, as a dangerous factor for cerebral apoplexy, coronary heart disease, myocardial infarction and sudden cardiac death, as well as an important dangerous factor for boosting hypertension, abnormal sugar tolerance and diabetes. Hyperlipoidemia may also result in fatty liver, liver cirrhosis, cholelithiasis, pancreatitis, hemorrhage of the ocular funcus, blindness, periphery blood vessel diseases, limp, hyperuricemia, etc. Therefore, the reduction of blood fat, as well as prophylaxis and treatment of cerebral apoplexy, coronary heart disease, myocardial infarction, sudden cardiac death, hypertension, abnormal sugar tolerance, diabetes, fatty liver, liver cirrhosis, cholelithiasis, pancreatitis, hemorrhage of the ocular funcus, blindness, periphery blood vessel diseases, limp, hyperuricemia, etc. should be planned as a whole, which should be supplemented each other.

Recently, according to the different main therapy effects, blood fat adjustments are classified into the following two sorts: statins such as simvastatin (Zocor), Pravastatin (Mevalotin) and fluvastatin, (Lescol) for mainly lowering total cholesterol and low density lipoprotein in the blood; and brates such as fenofibrate and gemfibrozil for mainly lowering triglyceride. These medicaments are major for the prophylaxis and treatment of abnormal blood fat, and they also have the effect on increasing in high density lipoprotein, so they have been widely used.

The side chain structure of statins has a portion similar to hydroxymethyl glutaryl coenzyme A (HMC-CoA), which can competitively inhibit the synthesis of cholesterol, so they can eliminate hyperlipoidemia and have a good effect on preventing cardiovascular and cerebrovascular diseases.

However, although statins have many positive therapy effects, it is reported that they also have many side effects (See “Life Extension Magazine”, November, 2004, Cholesterol & Statin Drugs Separating Hype from Reality, William Davis, MD). In recent years, a series of follow up monitoring for the use of medicine and further researches indicate that although only about 2% of the patients suffered from serious muscle damage and liver function obstacle, in many doctors' experiences of administration, it is found that about 30% of the patients who were given statins suffered from muscle pain and feel weak in different degrees. This is because that the increase in the concentration of statins in the blood plasma is relevant to the danger of muscle pain and muscle disease, especially, the risk of dissolution of striated muscle also increases. And, it is further found that statins may inhibit the synthesis of coenzyme Q10, and lower the ability of synthesizing coenzyme Q10 in the body per se, so as to greatly lower the concentration of coenzyme Q10 in the important organs in the body, such as blood vessel, kidney, liver and heart (See the report in http://www.cmt.com.cn/article/040401/a0404010601.htm), thus these organs are liable to pathologic changes. For example, when a patient with the abnormal heart function is given lovastatin (40 mg, Mevacor) alone, the ejection fraction of heart is greatly lowered (from 0.70 to 0.54 within 6 months), and when a patient suffering fatty liver is given statins, the condition of fatty liver will be worse, causing disordered liver function of the patient, etc.

Consequently, when statins are administrated alone, the patient, with normal organs, and having high level of blood fat, hypertension and high level of cholesterol, results in side effects such as muscle pain and muscle diseases, as well as the increasing danger of the dissolution of striated muscle. The administration of statins alone is also not suitable for the patient which has problems on organs such as blood vessel, kidney, livers and heart, however, these diseases usually cause the concentration of cholesterol in the blood of the patient increasing, so that the therapy measure to lower the concentration of cholesterol in the blood is needed.

For example, as reported by Yamamoto Y. and Yamashita S. et. al. (“Plasma ubiquinone to ubiquinol ratio in patients with hepatitis, cirrhosis, and hepatoma, and in patients treated with percutaneous transluminal coronary reperfusion”, Biofactors 1999; 9:241-246), one in vivo symptom of a liver disease patient is the serious lack of coenzyme Q10, so the liver disease patient suffers from not only lipid abnormity which causes heart diseases, but also other serious diseases such as kidney disease and cancer.

Moreover, the commonest lipid metabolic abnormity in the diabetes is the increased triglyceride and the reduced high density lipoprotein, and another serious chronic complication is kidney diseases. Therefore, to avoid the occurrence of cardiovascular and cerebrovascular diseases, it is important for diabetes to treat hyperlipoidemia and protect kidney besides of the good control of blood sugar.

The increase in the concentration of cholesterol in the blood is caused by a series of reasons, including pathologic changes of heart, kidney and livers. To actually eliminate the increasing of the concentration of cholesterol in the blood, it should be ensured that all of these causes of disease are eliminated. Therefore, it is very important for the healthy and high quality life of people by developing a therapy which can completely and effectively lower blood fat, protect blood vessels of heart and brain and provide them with nutrition, and protect and repair other organs which are damaged by the increasing of blood fat to some extent, such as kidney and liver, as well as has few or no obvious side effects. According to the principle of the complementation combination in the traditional Chinese medicine, the present inventors have developed a new multi-components formulation in low cost based on the existing small molecular chemical medicine which has been proved to be very effective.

### CONTENT OF THE INVENTION

(1) Object of the Invention

The main object of the present invention is to safely and reliably lower the concentration of cholesterol in the blood, so that patients with kidney disease and patients with liver disease can also use statins, and to lower, even to eliminate the side effects of statins on muscle and liver.

(2) Active Ingredients in the Multi-Components Formulations

The active ingredients in said new formulation as described in the present invention include two or more of the following three active ingredients:
(a) agents for lowering blood fat
(b) agents for protecting and repairing liver; and
(c) agents for protecting kidney, heart, blood vessel or muscle.

(a) Agents for Lowering Blood Fat

Agents for lowering blood fat used in the present invention are statins.

Statins, i.e. hydroxymethyl glutaryl coenzyme A reductase inhibitors (HMC-CoA-RI), are one of the most important findings in the later of 20^{th} century, which can lower total cholesterol in the blood by 25-35% and lower the low density lipoprotein in the blood by 30-40%, and have a good effect on preventing diseases of heart and brain. Preferable statins include, but not limited to, lovastatin (Mevacor), simvastatin (Zocor), Pravastatin (Mevalotin), atorvastatin (Lipitor), rosuvastatin (CRESTOR), fluvastatin (Lescol), pitavastatin (Livalo), huivastatin, or the mixtures thereof.

(b) Agents for Protecting and Repairing Liver

Agents for protecting and repairing liver used in the present invention include, but not limited to, silymarin, silybin, L-carnitine, or genipin.

Silymarin is a main active ingredient of Milk Thistle extracted from its seed. As early as two thousand years ago, European found that Milk Thistle had the effects on protecting liver and reinforcing liver, and Milk Thistle was popular among civilians. In 1949, Milk Thistle was the first time to be proved to have the effects on protecting liver for patients damaged by carbon tetrachloride and treating hepatitis in a clinical trial in Germany. In 1968, the main active ingredient extracted from the seed in Germany, i.e. silymarin, was further studied and was found that silymarin contained three different components, wherein the one had best effect of medicine was silybin.

After around thirty years of tube trials, animal trials, clinical trials and research, silymarin has been generally proved to have the effects on protecting liver, reinforcing liver and detoxicating. For example, under the following conditions: Amanita intoxication, the mortality of which was usually about 30-40%, however, a clinical trial having 60 Amanita intoxication patients proves that upon treated by silymarin, all survived; and the other as Acute Viral Hepatitis, wherein 42 of 77 persons were administered Placebo and the rest 35 persons were given silymarin, the result being that the average recovery period of control group was 43 days, while that of the patients administered silymarin was merely 29 days. Up to the present, Milk Thistle has been proved to possess the following important functions: preventing the liver function from damages caused by alcohol, drugs, chemical substances such as carbon tetrachloride, insecticide, the pollutants and radioactivity in the air, etc.; treating and preventing liver cirrhosis, and promoting regeneration of liver cells; preventing various hepatitis, and lowering the index of liver ferment; promoting the negotiability of bile to prevent gall stone; treating and preventing wild Amanita intoxication; and having certain effects on anti-inflammation, lowering cholesterol, and lowering blood pressure and blood sugar.

L-Carnitine: when the body lacks Carnitine, the oxidation of long chain fatty acid is hindered, causing excessive fat storing in the liver to result in fatty liver.

Natural crosslinking agent Genipin: it can inhibit the reproduction of the virus of hepatitis B, thus having the effect on treating hepatitis B (See Korean Patent Laid-open No. 94-1886).

Other medicaments used in the present invention to protect and repair liver include blood vessel endothelial cell growth factor (VEGF), Astragalus extract (See “Chinese medicine Report”, Sep. 1986, 11(9):47-9), vitamin C, folic acid, glucurolactone, Inosine, Ethacrynic acid, Piretanide, Bumetanide, etc.

(c) Agents for Protecting Kidney, Heart, Blood Vessel or Muscle

Agents for protecting kidney, heart, blood vessel or muscle used in the present invention include, but not limited to coenzyme Q10, lipoic acid, or vitamin E.

Coenzyme Q10 mainly exists in the heart, liver, kidney and pancreas of human body, the important functions of which include adjusting the growth of cell and maintaining the cell per se, as well as anti-oxidation. The anti-oxidation effect thereof can protect important organs such as heart, liver, kidney and pancreas by lowering the damage of free radical to the cells within these organs. The liver of human body can synthesize coenzyme Q10 by using Tyrosine and Phenylalanine of the protein, vitamins E, B1 and B6, and folic acid, so as to meet own's needs. Peter D. Mitchell, Ph. D (Edinburgh university, Scotland) found when the human cells were producing energy, coenzyme Q10 must exist. Other effects of coenzyme Q10, including protecting heart and blood vessel which are the most important, have been or are found continuously. In the 80s of last century, Langsjoen, M. D. first made clinical trial by using coenzyme Q10 as follows: administrating coenzyme Q10 to 19 patients which were close to death due to heart failure, as a result, these patients surprisingly becoming better, i.e. the enlarged heart was recovered to the original state, and the function of beat was improved to pump more blood to flow to the tissues preserved by end micro-blood vessels. The researchers of Texas University (USA), having studied for 6 years, found that the addition of coenzyme Q10 into the traditional therapy for treating heart diseases could make 75% of patients add “three-year persistence”. Compared with the traditional therapy which could make 25% of patients have “three-year persistence”, coenzyme Q10 obviously had important therapy effect on the treatment of heart disease.

Therefore, if people have sufficient coenzyme Q10, the immunologic function thereof will be improved and germs and viruses invaded into the body will be vanished; it also has prevention effect on certain fearful diseases such as cancer, chronic infection, prayer beads germs and AIDS, and the like.

As a medicament for protecting heart, Alpha-Lipoic Acid has the effect on lowering cholesterol in the blood (See “Alpha Lipoic Acid Breakthrough: The Superb Antioxidant That May Slow Aging, Repair Liver Damage, and Reduce the Risk of Cancer, Heart Disease, and Diabetes”, BURT BERKSON, 1998, Three River Press, New York, N.Y.).

Vitamin E also has a certain protection effect.

All of the above silymarin, L-Carnitine, Genipin, lipoic acid, glucurolactone, Inosine, Vitamin E, Vitamin C, folic acid and coenzyme Q10 are natural products, and the safety and the desired therapy effects in the present invention have been completely confirmed. No toxic and side effect is found even though individual component therein is used at higher amount. For example, the amount of silymarin is 200 mg/kg body weight/day, the amount of L-Carnitine is 500 mg/kg body weight/day, the amount of lipoic acid is 300 mg/day, the amount of Vitamin E is 500 mg/day, and the amount of coenzyme Q10 is 100 mg/kg body weight/day. In the most European and American countries and regions, the above products are sold in the form of nutrient or OTC medicine.

Up to now, no possible unfavored interaction is found between the above-mentioned active ingredients and statins, which can be confirmed in www.drugdigest.org/DD/Interaction which technically reports interactions of medicines.

(3) Pharmaceutical Composition

The pharmaceutical composition of the present invention usually comprises:
(a) 5-100 parts by weight of an agent for lowering blood fat;
(b) 20-1000 parts by weight of an agent for protecting and repairing liver;
(c) 20-500 parts by weight of an agent for protecting kidney, heart, blood vessel or muscle; and
(d) 50-2000 parts by weight of a pharmaceutically acceptable carrier or diluent.

In a preferred embodiment, the pharmaceutical composition of the present invention is made into unit dosage forms. In the unit dosage form, the amount of statins may be 0.1-100 mg/dosage, preferably 5-80 mg; the amount of coenzyme Q10 may be 0.1-500 mg/dosage, preferably 20-250 mg/dosage; or the amount of silymarin may be 0.1-1000 mg/dosage, preferably 20-500 mg/dosage.

More preferably, when the combined medicament is consisted of statins, coenzyme Q10 and silymarin, the ratio thereof is generally 1:1˜20:1˜50(or 1:1:1˜1:20:50), preferably 1:1˜10:1˜30. Generally, each dosage form comprises 5-80 mg statins, 50-200 mg coenzyme Q10, and 80-500 mg silymarin.

In addition to the components (a)-(d), the composition of the present invention further comprises optional additive, such as anti-oxidant, flavor-blocking agent, food color and pH regulator.

To better illustrate the present invention, the following description is based on the combination of simvastatin, coenzyme Q10 and silymarin.

(4) Administration Manner, Dosage Form and Formulation

The administration manner of the present invention is not specifically limited, and the new medicaments described therein are suitable to be all dosage forms, including injectable dosage form such as microsphere for injection and liposome for intravenous injection; nasal cavity and lung inhalation administration system such as nasal drop, aerosol, powder, gel, microsphere, microparticle, nanoparticle, liposome and emulsion; orally slow release and controlled release dosage form such as fluid slow release and controlled release formulation technique, compound slow release and controlled release formulation technique, constant speed release technique, constant location release technique and constant time release technique; and skin-permeation administration system such as membrane permeation technique, skeleton controlled release technique, micro-reservoir technique and adhesive separation technique, and the like. For those skilled in the art, they can prepare the new medicaments described in the present invention as the relevant dosage forms desired in the market. Therefore, the present invention encompasses all above-mentioned effective components and used dosage forms.

The amounts of the therapeutic effective components and administration solutions used for treating specific diseases by the present invention are dependent on various factors, including body weights, ages, genders, essential medical symptom, degree of the disease, and the manner and frequency of administration.

To better illustrate the present invention, the following description is based on the examples of two dosage forms, i.e. solid simvastatin tablet and simvastatin soft capsule.

The solid tablet dosage form in the present invention is based on simvastatin sale tablet (Zocor) dosage form produced by Merck, which is developed by optimizing combination. Zocor tablet produced by Merck is composed of a core and a shell coating membrane. The core comprises an active component simvastatin, a non-active component anhydrous lactose, a microcrystalline cellulose, a pregellatinized maize starch, a magnesium stearate, a butylated hydroxyanisol (BHA), a citric acid monohydrate and an ascorbic acid. The water-soluble coating membrane on the surface of the tablet comprises a hydroxypropyl cellulose, a methylhydroxypropyl cellulose, a talc, a titanium dioxide, a iron oxide red and a iron oxide yellow. Generally, lactose and cellulose are generally used as fillers and adhesives, and starch is used as a filler and a disintegrating agent. Since simvastatin is stable in an aqueous solution only in an acid condition, the function of citric acid and ascorbic acid is likely to ensure the acid environment within the tablet to keep the stable lactone molecular structure of simvastatin, so as to reduce or avoid the formation of simvastatin dimeric by-product. When 40 mg Zocor tablet is dissolved in pure water, the measured pH is 2.8, while the tablet having high acidity typically tends to be hydroscopic and deform under a high temperature. Therefore, strong acid should be avoided as an excipient. In addition, since magnesium stearate is used as a lubricant in the tablet, and metal ions such as magnesium has the effect of catalyzing and oxidizing carbon-carbon double bond, another effect of citric acid may be to complex with magnesium metal ion to lower its catalyzing and oxidizing effect.

Experiment on Stability of Simvastatin:

Zocor tablet was placed under the condition of 40° C., and the appearance at different time was viewed and the dissolution rate was measured. The dissolution rate was measured in an aqueous sodium phosphate buffer solution with pH of 7.0 by USP (United States Pharmacopoeia) Method II (dissolution test, method II, paddle). The test was conducted by experimental apparatus method II with the stirring rate of 50 rpm. The experimental solution was 900 mL 0.01 M sodium phosphate buffer solution with pH of 7.0, and the solution comprised 0.5% sodium lauryl sulfate. The standard method requested that the dissolution extent within 30 min be no less than 75%. After studying, we found that the new Zocor tablet (40 mg) without hyperthermic treatment had 98% of solid dissolution within 30 min, while the original packaged Zocor tablet, which was placed under 40° C. for 6 months, had only 18% of solid dissolution within 30 min. Furthermore, simvastatin solid had good stability under pH of 5-7. Under the condition that the non-aqueous solution comprised a solid dosage form, without additional acid, and the amounts of dimer and other degradation substance were not obviously increased, simvastatin was stable. This indicated that reducing or eliminating citric acid and ascorbic acid was feasible. Further, when the lactose was replaced with the microcrystalline cellulose as the main filler and adhesive in the formulation, for example, the weight amount in the solid dosage form went beyond 20% or 40%, even 60%, while the other components were kept unchanged, the stability of simvastatin increased. Another advantage of replacing lactose with the microcrystalline cellulose as the main filler and adhesive was that the microcrystalline cellulose could also be used as a disintegrating agent, the aqueous dissolution rate of the new formulation was quicker, and the amount of other disintegrating agent in the new formulation could be reduced.

Study on Oxidizing Stability of Simvastatin:

We found that when the lactose was replaced with the microcrystalline cellulose as the main filler and adhesive, or the magnesium stearate was replaced with zinc stearate as the lubricant, the anti-oxidant such as butylated hydroxyanisol, citric acid and vitamin C might not be added. At this time, the anti-oxidation property of the new simvastatin tablet was similar to that of the simvastatin sale medicine Zocor tablet of Merck. Furthermore, when simvastatin tablet included more than 50 mg coenzyme Q10 or more than 80 mg silymarin, the anti-oxidation property thereof was better than that of the simvastatin sale medicine Zocor tablet of Merck. The experiment on anti-oxidation property was to place the new tablet and Zocor tablet together under 60° C., and after 4 weeks, a high performance liquid chromatogram (HPLC) method was used to detect the amount of the oxidized simvastatin in the sample. According to our findings, in our invented new dosage form, the amounts of the above-mentioned excipients such as citric acid, ascorbic acid, lactose, butylated hydroxyanisol and magnesium stearate could be reduced or eliminated.

The gastrointestinal part in human body is a complex system, which can absorb water soluble substances and lipid substances. Since three main active ingredients in the present invention are fat soluble substances except pravastatin and huivastatin, the solubility of said components, especially silymarin and coenzyme Q10, in an aqueous medium is very low. Therefore, if there is no suitable ancillary substance, the re-absorption extent thereof in the digestive system is very low, and the corresponding bioavailability is also not good. The formulation method disclosed in the following text can well solve the problem of re-absorption, to make these effective components reach or be close to the desired bioavailability in the human body.

There are many methods to increase the re-absorption extent of fat soluble substance in the digestive system and increase the bioavailability thereof, such as the addition of lipid or fat soluble assistant substance, the addition of water diffusible assistant substance, or the addition of both. The conventional lipid or fat soluble assistant substance includes tocopherol (vitamin E), plant oil, lecithin, etc. The conventional water soluble and water diffusible assistant substance includes lecithin, polysorbate-80, oil lactic acid, etc. The amount of lipid or fat soluble assistant substance is 0-98% by weight, typically 5-60% by weight, preferably 10-40% by weight, most preferably about 20% by weight. The amount of water soluble and water diffusible assistant substance is 0-98% by weight, typically 10-60% by weight, preferably 3-30% by weight, most preferably 7-30% by weight.

Consequently, anhydrous lactose can be used in the tablets of the present invention as the filler and the adhesive. It is much better to use a microcrystalline cellulose as filler and adhesive, as well as the disintegrating agent; to use sodium starch glycolate, crospovidone and pregellatinized maize starch as the disintegrating agent; to use zinc stearate and Sodium Stearyl Fumarate as the lubricant; to use butylated hydroxyanisol as the anti-oxidant or not to use it; and to use tocopherol and lecithin as lipid or fat soluble assistant substance, to use lecithin and polysorbate-80 as water soluble and water diffusible assistant substance, or both together.

The tablet of the present invention can also comprise a surface coating membrane. The components of the coating membrane can comprise hydroxypropyl cellulose, methylated hydroxypropyl cellulose, talc, titanium dioxide and other coloring agents.

(5) Preparation Method

The production of the pharmaceutical composition of the present invention can be conducted by various techniques in the prior art.

As to the tablets, generally, simvastatin, coenzyme Q10, silymarin and one or two excipients are first mixed into a mixture by wet process or dry process. The mixing process can be granulating, slugging, blending, etc. If the formulation comprises a butylated hydroxyanisol, it is usually needed to first dissolve the butylated hydroxyanisol into a solvent, then mix the butylated hydroxyanisol solution with excipients, and dry together; or to first uniformly mix the butylated hydroxyanisol and one main excipient such as microcrystalline cellulose, then mix with the active ingredients simvastatin, coenzyme Q10, silymarin and the other excipients. The uniformly mixed mixture is made into a tablet by compressing.

When the plant oil, such as palm oil, coconut oil, palm fruit oil, palm stearine oil, coffee oil, soybean oil, safflower oil, canola oil, grape seed oil, cotton seed oil, corn oil, sunflower seed oil, sesame oil, olive oil, barley oil, quinoa oil, castor oil, peanut oil and rape seed oil, is used as the lipid or fat soluble ancillary substance, the formulation of tablet will be difficult. In such a case, the use of soft capsule formulation is desired.

As to soft capsule, generally, simvastatin, coenzyme Q10, silymarin, lipid or fat soluble assistant substance, and one or two excipients are first mixed into a mixture. If the formulation comprises butylated hydroxyanisol, it should be first dissolved in a solvent, then mix the butylated hydroxyanisol solution with excipients; or to first uniformly mix the butylated hydroxyanisol with one main excipients such as microcrystalline cellulose, then mix with the active ingredients simvastatin, coenzyme Q10, silymarin and the other excipients. The uniformly mixed mixture is made into a soft capsule.

(6) Beneficial Effects

The new medicament in the present invention can lower the level of cholesterol in the patient, and meanwhile, ensure that the amount of coenzyme Q10 in the body of the patient is normal. Compared with the patient taking statins alone, essentially no symptom of muscle pain or feeling weak appears, let alone serious muscle damage and liver function obstacle. Especially, for diabetics that has serious chronic complication such as kidney disease, through taking the medicine, the symptom is obviously improved. For the patient who has hepatomegaly, the liver edema, fatty liver and hepatitis, the liver function thereof is obviously improved.

Compared with Zocor tablet sold in the market, the new medicine and dosage form in the present invention cut down some unnecessary excipients, and the production process is simpler and more convenient.

### MODE OF CARRYING OUT THE INVENTION

The present invention will be further illustrated in combination with the following specific examples. It should be understood that, these examples are exemplary only, not intended to limit the extent of the present invention. The experimental methods in the following examples which not indicate the specific experimental conditions are typically carried out under conventional conditions, or following the manufacture's instructions.

### EXAMPLE 1

The samples in the following table included various formulations. These formulations could be used in tablet and soft capsule.

The typical production method of tablet was as follows:
1. Firstly, butylated hydroxyanisol was uniformly mixed with one main excipients such as microcrystalline cellulose in a ratio of 1:10.
2. Simvastatin, coenzyme Q10, silymarin and the other excipients were uniformly mixed with the mixture obtained in the above step 1.
3. The uniform mixture obtained in the above step 2 was further pressed into small particles. 4. The lubricant zinc stearate, talc and the small particles obtained in the above step 3 were mixed for several minutes.
5. The uniform mixture obtained in the above step 4 was compressed into a tablet.
6. If necessary, the tablet obtained in the above step 5 was coated by misty droplets sprayed from a coating liquid.

If a soft capsule formulation was used, the uniform mixture obtained in the above step 2 was directly molded into a soft capsule.

TABLE 1
(Unit: mg)

| | Sample | Sample | Sample | Sample | Sample | Sample |
|---|---|---|---|---|---|---|
| Component | 1-1 | 1-2 | 1-3 | 1-4 | 1-5 | 1-6 |
| simvastatin | 5 | 10 | 20 | 40 | 5 | 5 |
| coenzyme Q10 | 50 | 50 | 50 | 100 | 100 | 100 |
| silymarin | 80 | 80 | 150 | 80 | 150 | 150 |
| micro- | 48 | 48 | 9.9 | 9.8 | 9.9 | 10 |
| crystalline | | | | | | |
| cellulose | | | | | | |
| crospovidone | 15 | 15 | 0 | 50 | 0 | 50 |
| butylated | 0.1 | 0.1 | 0.1 | 0.1 | 0 | 0 |
| hydroxyanisol | | | | | | |
| pregellatinized | 10 | 10 | 9.8 | 8.2 | 9.9 | 8.2 |
| maize starch | | | | | | |
| zinc stearate | 0.2 | 0.2 | 0.2 | 0.2 | 0.2 | 0.2 |
| talc | 1.7 | 1.7 | 0 | 1.7 | 0 | 2.0 |
| tocopherol | 50 | 0 | 50 | 0 | 50 | 0 |
| palm oil | 0 | 0 | 50 | 0 | 50 | 0 |

### EXAMPLE 2

Samples 2-1 to 2-4 were obtained based on the procedure of Example 1, except that the formulations illustrated in Table 2 were used.

TABLE 2
(Unit: mg)

| | Sample | Sample | Sample | Sample |
|---|---|---|---|---|
| Component | 2-1 | 2-2 | 2-3 | 2-4 |
| simvastatin | 20 | 40 | 10 | 10 |
| coenzyme Q10 | 50 | 100 | 100 | 100 |
| silymarin | 150 | 80 | 150 | 150 |
| microcrystalline | 9.9 | 10 | 9.9 | 10 |
| cellulose | | | | |
| crospovidone | 0 | 50 | 0 | 50 |
| butylated hydroxyanisol | 0.1 | 0.1 | 0 | 0 |
| pregellatinized maize | 9.8 | 0 | 9.9 | 0 |
| starch | | | | |
| zinc stearate | 0.2 | 0.2 | 0.2 | 0.2 |
| talc | 0 | 1.7 | 0 | 1.8 |
| polysorbate-80 | 48 | 56 | 56 | 60 |

### EXAMPLE 3

Samples 3-1 to 3-6 were obtained based on the procedure of Example 1, except that the formulations illustrated in Table 3 were used.

TABLE 3
(Unit: mg)

| | Sample | Sample | Sample | Sample | Sample | Sample |
|---|---|---|---|---|---|---|
| Component | 3-1 | 3-2 | 3-3 | 3-4 | 3-5 | 3-6 |
| simvastatin | 10 | 20 | 20 | 20 | 80 | 80 |
| coenzyme Q10 | 100 | 100 | 100 | 100 | 200 | 200 |
| silymarin | 120 | 240 | 360 | 500 | 240 | 240 |
| micro- | 60 | 60 | 80 | 80 | 80 | 80 |
| crystalline | | | | | | |
| cellulose | | | | | | |
| crospovidone | 15 | 15 | 30 | 50 | 30 | 50 |
| butylated | 0.2 | 0.2 | 0.2 | 0.2 | 0.2 | 0.2 |
| hydroxyanisol | | | | | | |
| pregellatinized | 18 | 18 | 18 | 18 | 18 | 18 |
| maize starch | | | | | | |
| zinc stearate | 0.2 | 0.2 | 0.2 | 0.2 | 0.2 | 0.2 |
| talc | 1.6 | 1.6 | 2.6 | 2.6 | 2.6 | 2.6 |
| tocopherol | 50 | 0 | 0 | 0 | 0 | 0 |

### EXAMPLE 4

### Animal Test

The animal test employed SD (Sprague-Dawley) male rats with the average body weight of 250 g. The experimental method was once oral administration of the combination with the highest amounts, i.e. the ratio of lovastatin, silymarin and coenzyme Q10 in the combination was 1.5:200:50 mg/kg body weight/day; the ratio of simvastatin, silymarin and coenzyme Q10 in the combination was 2.0:200:50 mg/kg body weight/day; the ratio of simvastatin, L-carnitine and coenzyme Q10 in the combination was 2.0:500:50 mg/kg body weight/day; the ratio of simvastatin, vitamin E and coenzyme Q10 in the combination was 2.0:8.0:50 mg/kg body weight/day; and the ratio of simvastatin, L-carnitine and vitamin E in the combination was 2.0:500:8.0 mg/kg body weight/day. Each combination fed three rats.

None of the compound compositions displayed toxic, side effect or discomfort. After 4 weeks, no rat died and the growth of body weight was not affected, thus indicating that these combinations were safe after use during the long period. When the animal test was finished, the results of blood tests of the test group and the control group displayed that the haematic crasis, serum glucose levels and Blood Urea Nitrogen (BUN) of two groups was consistent. And, the results of anatomy tests of the main organs also showed that the organs of the administration group were normal.

### EXAMPLE 5

### Summary of Clinical Cases

1. The patient B. C., 62 years old, white, male; before using simvastatin (20 mg, ZOCOR, Merck), the concentration of cholesterol in the blood being 249 mg/L; ischemic cardiomyopathy; cardiac functional capacity being Class III (minimal activity causes distress, New York Heart Association); ejection fraction being 0.59; moderate hepatic fibrosis; the concentration of blood ammonia being 72 μg/L; the concentration of coenzyme Q10 in the blood being 1.84 μg/mL; and the liver detection employing acupuncture liver biopsy, merely twice, i.e. before test and after test.

After one-month use of simvastatin (20 mg), the patient B. C. felt muscle anergy and aching pain, and the concentration of cholesterol in the blood became 202 mg/L. After 6 months, the muscle anergy and aching pain was further exacerbated, the concentration of cholesterol in the blood became 189 mg/L, ejection fraction was 0.61, the concentration of coenzyme Q10 in the blood was 1.12 μg/mL, hepatic fibrosis became depth hepatic fibrosis, the symptom of liver cirrhosis appeared, the concentration of blood ammonia was 162 μg/L, and cardiac functional capacity was still Class III.

This indicated that statins might damage liver when lowering the concentration of coenzyme Q10 in the blood, so the doctor had to stop the administration.

Summary of the case of the patient B. C.

| | | After test | | | | | | |
|---|---|---|---|---|---|---|---|---|
| | Before | 5 | 10 | 15 | 20 | 25 | 30 | 35 |
| | test | weeks | weeks | weeks | weeks | weeks | weeks | weeks |
| coenzyme Q10 | 1.84 | 1.72 | 1.57 | 1.44 | 1.38 | 1.28 | 1.18 | 1.12 |
| (μg/ml) | | | | | | | | |
| cholesterol in the | 249 | 202 | 198 | 194 | 197 | 192 | 194 | 189 |
| blood (mg/l) | | | | | | | | |
| ejection | 0.59 | 0.62 | 0.60 | 0.63 | 0.58 | 0.62 | 0.61 | 0.61 |
| fraction | | | | | | | | |
| blood ammonia | 72 | 84 | 69 | 77 | 108 | 89 | 93 | 162 |
| (μg/l) | | | | | | | | |

2. The patient H. G., 61 years old, white, male; before using the combined medicament, the concentration of cholesterol in the blood being 255 mg/L; ischemic cardiomyopathy; cardiac functional capacity being Class III; ejection fraction being 0.60; moderate hepatic fibrosis; the concentration of blood ammonia being 79 μg/L; the concentration of coenzyme Q10 in the blood being 2.32 μg/mL.

After taking the combined medicament indicated in Sample 1-3 of the table in Example 1 for 6 months (the administration amount was 1 tablet/day, and the following cases 3-5 were the same), the concentration of cholesterol in the blood became 191 mg/L, ejection fraction was increased to 0.73, the concentration of coenzyme Q10 in the blood was 2.24 μg/mL, no feeling of muscle discomfort appeared, moderate hepatic fibrosis transferred into gentle hepatic fibrosis, the concentration of blood ammonia was always kept at the normal range of 50-81 μg/L after two weeks of administration, and cardiac functional capacity transferred into Class I (ordinary activity causes no discomfort, New York Heart Association).

This indicated that the compound formulation of the present invention could lower the cholesterol in the blood, and reverse the hepatic fibrosis when improving cardiac functional capacity.

Summary of the case of the patient H. G.

| | | After test | | | | | | |
|---|---|---|---|---|---|---|---|---|
| | Before | 5 | 10 | 15 | 20 | 25 | 30 | 35 |
| | test | weeks | weeks | weeks | weeks | weeks | weeks | weeks |
| coenzyme Q10 | 2.32 | 2.28 | 2.35 | 2.33 | 2.19 | 2.28 | 2.31 | 2.24 |
| (μg/ml) | | | | | | | | |
| cholesterol in the | 255 | 194 | 198 | 186 | 187 | 182 | 184 | 191 |
| blood (mg/1) | | | | | | | | |
| ejection | 0.60 | 0.68 | 0.72 | 0.69 | 0.68 | 0.72 | 0.71 | 0.73 |
| fraction | | | | | | | | |
| blood ammonia | 79 | 54 | 50 | 77 | 81 | 59 | 63 | 62 |
| (μg/1) | | | | | | | | |

3. The patient X. C., 39 years old, Asian, male; before using the combined medicament, the concentration of cholesterol in the blood being 259 mg/L; moderate non-alcohol fatty liver; and the concentration of coenzyme Q10 in the blood being 2.62 μg/mL.

After taking the combined medicament indicated in Sample 1-5 of the table in Example 1 for 6 months, the concentration of cholesterol in the blood became 178 mg/L, the concentration of coenzyme Q10 in the blood was 2.54 μg/mL, no feeling of muscle discomfort appeared, and the symptom of fatty liver disappeared.

This indicated that the combined formulation of the present invention might lower the cholesterol in the blood, and treat fatty liver while avoiding the muscle anergy and aching pain caused by the administration of statins.

Summary of the case of the patient X. C.

| | | After test | | | | | | |
|---|---|---|---|---|---|---|---|---|
| | Before | 5 | 10 | 15 | 20 | 25 | 30 | 35 |
| | test | weeks | weeks | weeks | weeks | weeks | weeks | weeks |
| coenzyme Q10 | 2.62 | 2.72 | 2.57 | 2.44 | 2.58 | 2.38 | 2.48 | 2.54 |
| (μg/ml) | | | | | | | | |
| cholesterol in the | 259 | 202 | 188 | 194 | 191 | 182 | 184 | 178 |
| blood (mg/l) | | | | | | | | |

4. The patient C. J., 46 years old, Asian, female; before using the combined medicament, the concentration of cholesterol in the blood being 256 mg/L; moderate non-alcohol fatty liver; and the concentration of coenzyme Q10 in the blood being 2.60 μg/mL.

After taking the combined medicament indicated in Sample 2-1 of the table in Example 2 for 6 months, the concentration of cholesterol in the blood became 181 mg/L, the concentration of coenzyme Q10 in the blood was 2.56 μg/mL, no feeling of muscle discomfort appeared, and the symptom of fatty liver disappeared.

This indicated that the combined formulation of the present invention could lower the cholesterol in the blood, and treat fatty liver (for both male and female) while avoiding the muscle anergy and aching pain caused by the administration of statins.

Summary of the case of the patient C. J.

| | | After test | | | | | | |
|---|---|---|---|---|---|---|---|---|
| | Before | 5 | 10 | 15 | 20 | 25 | 30 | 35 |
| | test | weeks | weeks | weeks | weeks | weeks | weeks | weeks |
| coenzyme Q10 | 2.60 | 1.72 | 1.57 | 1.44 | 1.38 | 1.28 | 1.18 | 2.56 |
| (μg/ml) | | | | | | | | |
| cholesterol in the | 256 | 201 | 195 | 194 | 196 | 182 | 178 | 181 |
| blood (mg/l) | | | | | | | | |

5. The patient L. S., 46 years old, white, female; before the administration of the combined medicament, the concentration of cholesterol in the blood being 251 mg/L; diabetic; diabetes and kidney disease early stage; and the concentration of coenzyme Q10 in the blood being 1.87 μg/mL.

After taking the combined medicament indicated in Sample 1-2 of the table in Example 1 for 6 months, the concentration of cholesterol in the blood became 181 mg/L, the concentration of coenzyme Q10 in the blood was 2.36 μg/mL, no feeling of muscle discomfort appeared, and the symptom of the thickening of glomerule capillary basilar membrane disappeared.

This indicated that the combined formulation of the present invention could lower the cholesterol in the blood, and had good effect on treating some chronic complication of diabetes, while avoiding the muscle anergy and aching pain caused by taking statins.

Summary of the case of the patient L. S.

| | | After test | | | | | | |
|---|---|---|---|---|---|---|---|---|
| | Before | 5 | 10 | 15 | 20 | 25 | 30 | 35 |
| | test | weeks | weeks | weeks | weeks | weeks | weeks | weeks |
| coenzyme Q10 | 1.87 | 2.12 | 2.23 | 2.19 | 2.38 | 2.28 | 2.33 | 2.36 |
| (μg/ml) | | | | | | | | |
| cholesterol in the | 251 | 206 | 193 | 194 | 186 | 182 | 183 | 181 |
| blood (mg/l) | | | | | | | | |

All publications, patents and patent applications mentioned in this specification are herein incorporated by reference into the specification to the same extent as if each individual publication, patent or patent application is specifically and individually indicated to be incorporated herein by reference. Additionally, it will be understood that in light of the above disclosure of the present invention, those skilled in the art can make various changes and modifications, and these equivalents fall within the scope of the claims of the present application as attached.

1. A pharmaceutical composition for lowering blood fat, which comprises:
(a) an agent for lowering blood fat;
(b) an agent for protecting and repairing liver;
(c) an agent for protecting kidney, heart, blood vessel or muscle; and
(d) a pharmaceutically acceptable carrier.

2. The pharmaceutical composition of claim 1, wherein the agent for lowering blood fat is statins;
the agent for protecting and repairing liver is selected from the group consisting of silymarin, L-carnitine, genipin, blood vessel endothelial cell growth factor, Astragalus extract, vitamin C, folic acid, glucurolactone, Inosine, Ethacrynic acid, Piretanide, Bumetanide, or the combination thereof; and
the agent for protecting kidney, heart, blood vessel or muscle is selected from the group consisting of coenzyme Q10, lipoic acid, vitamin E, or the combination thereof.

3. The pharmaceutical composition of claim 1, which comprises:
(a) 5-100 parts by weight of the agent for lowering blood fat;
(b) 20-1000 parts by weight of the agent for protecting and repairing liver;
(c) 20-500 parts by weight of the agent for protecting kidney, heart, blood vessel or muscle; and
(d) 50-2000 parts by weight of the pharmaceutically acceptable carrier.

4. The pharmaceutical composition of claim 1, wherein the components (a), (b) and (c) are statins, silymarin, and coenzyme Q10, respectively.

5. The pharmaceutical composition of claim 4, wherein the ratio of statins, coenzyme Q10 and silymarin is 1:1˜20:1˜50.

6. The pharmaceutical composition of claim 4, which is a single dosage form, and each dosage comprises 0.1-100 mg statins, 0.1-1000 mg silymarin and 0.1-500 mg coenzyme Q10.

7. The pharmaceutical composition of claim 6, wherein the statins are selected from the group consisting of lovastatin, simvastatin, Pravastatin, atorvastatin, rosuvastatin, fluvastatin, pitavastatin, huivastatin, or a mixture thereof.

8. The pharmaceutical composition of claim 1, which is a tablet or a soft capsule.

9. The pharmaceutical composition of claim 1, wherein the pharmaceutically acceptable carrier includes lipids or fat soluble assistant substance, a water diffusible ancillary substance, or the combination thereof.

10. A method for producing a pharmaceutical composition, which comprises the following step:
mixing the following components to obtain the composition
(a) 5-100 parts by weight of an agent for lowering blood fat;
(b) 20-1000 parts by weight of an agent for protecting and repairing liver;
(c) 20-500 parts by weight of an agent for protecting kidney, heart, blood vessel or muscle; and
(d) 50-2000 parts by weight of a pharmaceutically acceptable carrier or diluent.
